(19) United States
(12) Patent Application Publication
(10) Pub. No.: US 2015/0106028 A1
(43) Pub. Date: Apr. 16, 2015

(54) CHARACTERIZATION OF CRUDE OIL BY FOURIER TRANSFORM ION CYCLOTRON RESONANCE MASS SPECTROMETRY

(71) Applicants: Omer Refa KOSEOGLU, Dhahran (SA); Adnan Al-Hajji, Dammam (SA); Hendrik Muller, Dhahran (SA); Hanadi H. Jawad, Qatif (SA)

(72) Inventors: Omer Refa KOSEOGLU, Dhahran (SA); Adnan Al-Hajji, Dammam (SA); Hendrik Muller, Dhahran (SA); Hanadi H. Jawad, Qatif (SA)

(21) Appl. No.: 13/467,693

(22) Filed: May 9, 2012

Related U.S. Application Data
(60) Provisional application No. 61/502,385, filed on Jun. 29, 2011.

(51) Int. Cl. H01J 49/00; G01N 33/28; H01J 49/38; G06F 19/00

(52) U.S. Cl. CPC H01J 49/0036; G01N 33/2811; H01J 49/38
USPC 702/23; 702/25

(57) ABSTRACT

A system, method and computer program product are provided for calculating the cetane number, octane number, pour point, cloud point and aniline point of crude oil fractions from the density and Fourier transform ion cyclotron resonance mass spectrometry (FT-ICR MS) of a sample of the crude oil.

### FIELD OF THE INVENTION

This invention relates to a method and process for the evaluation of samples of crude oil and its fractions by Fourier transform ion cyclotron resonance mass spectrometry (FT-ICR MS), avoiding the need to conduct crude oil assays.

### BACKGROUND OF THE INVENTION

Crude oil originates from the decomposition and transformation of aquatic, mainly marine, living organisms and/or land plants that became buried under successive layers of mud and silt some 15-500 million years ago. They are essentially very complex mixtures of many thousands of different hydrocarbons. Depending on the source, the oil predominantly contains various proportions of straight and branched-chain paraffins, cycloparaffins, and naphthenic, aromatic, and polynuclear aromatic hydrocarbons. These hydrocarbons can be gaseous, liquid, or solid under normal conditions of temperature and pressure, depending on the number and arrangement of carbon atoms in the molecules.

Crude oils vary widely in their physical and chemical properties from one geographical region to another and from field to field. Crude oils are usually classified into three groups according to the nature of the hydrocarbons they contain: paraffinic, naphthenic, asphaltic, and their mixtures. The differences are due to the different proportions of the various molecular types and sizes. One crude oil can contain mostly paraffins, another mostly naphthenes. Whether paraffinic or naphthenic, one can contain a large quantity of lighter hydrocarbons and be mobile or contain dissolved gases; another can consist mainly of heavier hydrocarbons and be highly viscous, with little or no dissolved gas. Crude oils can also include heteroatoms containing sulfur, nitrogen, nickel, vanadium and other elements in quantities that impact the refinery processing of the crude oil fractions. Light crude oils or condensates can contain sulfur in concentrations as low as 0.01 W %; in contrast, heavy crude oils can contain as much as 5-6 W %. Similarly, the nitrogen content of crude oils can range from 0.001-1.0 W %.

The nature of the crude oil governs, to a certain extent, the nature of the products that can be manufactured from it and their suitability for special applications. A naphthenic crude oil will be more suitable for the production of asphaltic bitumen, a paraffinic crude oil for wax. A naphthenic crude oil, and even more so an aromatic one, will yield lubricating oils with viscosities that are sensitive to temperature. However, with modern refining methods there is greater flexibility in the use of various crude oils to produce many desired type of products.

A crude oil assay is a traditional method of determining the nature of crude oils for benchmarking purposes. Crude oils are subjected to true boiling point (TBP) distillations and fractionations to provide different boiling point fractions. The crude oil distillations are carried out using the American Standard Testing Association (ASTM) Method D 2892. The common fractions and their nominal boiling points are given in Table 1.

TABLE 1

| Fraction | Boiling Point, ° C. |
|---|---|
| Methane | −161.5 |
| Ethane | −88.6 |
| Propane | −42.1 |
| Butanes | −6.0 |
| Light Naphtha | 36-90 |
| Mid Naphtha | 90-160 |
| Heavy Naphtha | 160-205 |
| Light gas Oil | 205-260 |
| Mid Gas Oil | 260-315 |
| Heavy gas Oil | 315-370 |
| Light Vacuum Gas Oil | 370-430 |
| Mid Vacuum Gas Oil | 430-480 |
| Heavy vacuum gas oil | 480-565 |
| Vacuum Residue | 565+ |

The yields, composition, physical and indicative properties of these crude oil fractions, where applicable, are then determined during the crude assay work-up calculations. Typical compositional and property information obtained from a crude oil assay is given in Table 2.

TABLE 2

| | | Property | |
|---|---|---|---|
| Property | Unit | Type | Fraction |
| Yield Weight and | W % | Yield | All |
| Volume % | | | |
| API Gravity | ° | Physical | All |
| Viscosity | ° | Physical | Fraction boiling >250° C. |
| Kinematic @ 38° C. | | | |
| Refractive | Unitless | Physical | Fraction boiling <400° C. |
| Index @ 20° C. | | | |
| Sulfur | W % | Composition | All |
| Mercaptan Sulfur, | W % | Composition | Fraction boiling <250° C. |
| W % | | | |
| Nickel | ppmw | Composition | Fraction boiling >400° C. |
| Nitrogen | ppmw | Composition | All |
| Flash Point, COC | ° C. | Indicative | All |
| Cloud Point | ° C. | Indicative | Fraction boiling >250° C. |
| Pour Point, | ° C. | Indicative | Fraction boiling >250° C. |
| (Upper) | | | |
| Freezing Point | ° C. | Indicative | Fraction boiling >250° C. |
| Microcarbon | W % | Indicative | Fraction boiling >300° C. |
| Residue | | | |
| Smoke Point, mm | mm | Indicative | Fraction boiling between |
| | | | 150-250 |
| Octane Number | Unitless | Indicative | Fraction boiling <250° C. |
| Cetane Index | Unitless | Indicative | Fraction boiling between |
| | | | 150-400 |
| Aniline Point | ° C. | Indicative | Fraction boiling <520° C. |

Due to the number of distillation cuts and the number of analyses involved, the crude oil assay work-up is both costly and time consuming.

In a typical refinery, crude oil is first fractionated in the atmospheric distillation column to separate sour gas and light hydrocarbons, including methane, ethane, propane, butanes and hydrogen sulfide, naphtha (36°−180° C.), kerosene (180°−240° C.), gas oil (240°−370° C.) and atmospheric residue (>370° C.). The atmospheric residue from the atmospheric distillation column is either used as fuel oil or sent to a vacuum distillation unit, depending on the configuration of the refinery. The principal products obtained from vacuum distillation are vacuum gas oil, comprising hydrocarbons boiling in the range 370°−520° C., and vacuum residue, comprising hydrocarbons boiling above 520° C. The crude assay data help refiners to understand the general composition of the crude oil fractions and properties so that the fractions can be processed most efficiently and effectively in an appropriate refining unit. Indicative properties are used to determine the engine/fuel performance or usability or flow characteristic or composition. A summary of the indicative properties and their determination methods with description are given below.

The cetane number of diesel fuel oil, determined by the ASTM D613 method, provides a measure of the ignition quality of diesel fuel; as determined in a standard single cylinder test engine; which measures ignition delay compared to primary reference fuels. The higher the cetane number; the easier the high-speed; direct-injection engine will start; and the less white smoking and diesel knock after start-up are. The cetane number of a diesel fuel oil is determined by comparing its combustion characteristics in a test engine with those for blends of reference fuels of known cetane number under standard operating conditions. This is accomplished using the bracketing hand wheel procedure which varies the compression ratio (hand wheel reading) for the sample and each of the two bracketing reference fuels to obtain a specific ignition delay, thus permitting interpolation of cetane number in terms of hand wheel reading.

The octane number, determined by the ASTM D2699 or D2700 methods, is a measure of a fuel's ability to prevent detonation in a spark ignition engine. Measured in a standard single-cylinder; variable-compression-ratio engine by comparison with primary reference fuels. Under mild conditions, the engine measures research octane number (RON), while under severe conditions, the engine measures motor octane number (MON). Where the law requires posting of octane numbers on dispensing pumps, the antiknock index (AKI) is used. This is the arithmetic average of RON and MON, (R+M)/2. It approximates the road octane number, which is a measure of how an average car responds to the fuel.

The cloud point, determined by the ASTM D2500 method, is the temperature at which a cloud of wax crystals appears when a lubricant or distillate fuel is cooled under standard conditions. Cloud point indicates the tendency of the material to plug filters or small orifices under cold weather conditions. The specimen is cooled at a specified rate and examined periodically. The temperature at which cloud is first observed at the bottom of the test jar is recorded as the cloud point. This test method covers only petroleum products and biodiesel fuels that are transparent in 40 mm thick layers, and with a cloud point below 49° C.

The pour point of petroleum products, determined by the ASTM D97 method, is an indicator of the ability of oil or distillate fuel to flow at cold operating temperatures. It is the lowest temperature at which the fluid will flow when cooled under prescribed conditions. After preliminary heating, the sample is cooled at a specified rate and examined at intervals of 3° C. for flow characteristics. The lowest temperature at which movement of the specimen is observed is recorded as the pour point.

The aniline point, determined by the ASTM D611 method, is the lowest temperature at which equal volumes of aniline and hydrocarbon fuel or lubricant base stock are completely miscible. A measure of the aromatic content of a hydrocarbon blend is used to predict the solvency of a base stock or the cetane number of a distillate fuel. Specified volumes of aniline and sample, or aniline and sample plus n-heptane, are placed in a tube and mixed mechanically. The mixture is heated at a controlled rate until the two phases become miscible. The mixture is then cooled at a controlled rate and the temperature at which two separate phases are again formed is recorded as the aniline point or mixed aniline point.

To determine these properties of gas oil or naphtha fractions conventionally, these fractions have to be distilled from the crude oil and then measured/identified using various analytical methods that are laborious, costly and time-consuming.

Fourier transform ion cyclotron resonance mass spectrometry (FT-ICR MS) includes two components: an ionization source and a mass analyzer. The ionization source ionizes molecules, while the mass analyzer determines the mass-to-charge ratio (m/z) of ions.

A number of ionization sources have been used in gas chromatography and mass spectrometry, with some being preferable for gases, others for liquids, and others for solids. Ionization sources for gas chromatography include electron ionization (ED, which uses a glowing filament, which may break down the molecules under study. Inductively coupled plasma ionization (ICP) is a destructive technique which applies heat to reduce a sample to its atomic components. Chemical ionization (CI), a subset of EI, adds gases such as methane, isobutane, or ammonia, producing results that are less damaging to the molecules under study. Direct analysis in real time (DART) ionizes samples at atmospheric pressure using an electron beam. Matrix-assisted, laser desorption ionization (MALDI) is a solid phase process that uses laser energy to ionize molecules off a metal target plate. Electrospray ionization (ESI), is a liquid phase process that produces a fine mist of droplets, as from an atomizer.

FT-ICR MS frequently relies on ESI or on a related variant, such as atmospheric pressure chemical ionization (APCI) or atmospheric pressure photoionization (APPI). APCI uses a corona discharge from an electrified needle to induce ionization of a solvent, which in turn reacts with the sample molecules to induce a chemical reaction resulting in an ionized sample molecule. APPI uses a photon discharge from high-intensity ultraviolet light to ionize the solvent gas, which in turn ionizes the sample molecules. APCI works well with relatively small, neutral, or hydrophobic compounds, such as steroids, lipids, and non-polar drugs. APPI works well with highly non-polar molecules like napthols and anthracenes.

Thus, in the petroleum industry, FT-ICR is conducted using ESI, and preferably the APPI variant of ESI. A petroleum sample is diluted in an appropriate solvent and infused into the spectrometer. The liquid sample is evaporated and the components are ionized by ESI or APPI, yielding unfragmented gas phase ions of the sample components. These ions are trapped in the strong magnetic field of the mass analyzer, where their mass-to-charge ratios are determined with high resolution and accuracy. The spectrometer provides a resolution of R>300,000 at m/z 400, which is high enough for routinely separating signals spaced as closely as 3.4 mDa (SH₄ vs. ¹²C₃), which is essential for the correct assignment of the elemental composition (C_{c}HₕNₙOₒSₛNiᵢVᵥ) corresponding to each mass signal in petroleum samples. The identified elemental compositions are then classified according to the heteroatoms in their elemental composition, e.g., pure hydrocarbons, mono-sulfur (or mono-nitrogen) species for molecules with one sulfur (or nitrogen) atom, or molecules with any combination of heteroatoms. The corresponding double bond equivalent (DBE) values and carbon numbers are calculated for each identified elemental composition, where the DBE is defined as half the number of hydrogen atoms lacking from a completely saturated molecule with an otherwise identical number of carbon and heteroatoms.

Any new rapid, direct method to help better understand the crude oil composition and properties from the analysis of whole crude oil will save producers, marketers, refiners and/or other crude oil users substantial expense, effort and time. Therefore, a need exists for an improved system and method for determining the properties of crude oil fractions from different sources and classifying the crude oil fractions based on their boiling point characteristics and/or properties.

### SUMMARY OF THE INVENTION

The above objects and further advantages are provided by the present invention which broadly comprehends a system and a method for determining the indicative properties of a hydrocarbon sample. In accordance with the invention, indicative properties (i.e., cetane number, pour point, cloud point and aniline point of gas oil fraction and octane number of gasoline fraction in crude oils) are predicted by density and FT-ICR MS measurement of crude oils. The correlations also provide information about the gas oil properties without fractionation/distillation (crude oil assays) and will help producers, refiners, and marketers to benchmark the oil quality and, as a result, valuate the oils without performing the customary extensive and time-consuming crude oil assays.

### BRIEF DESCRIPTION OF THE DRAWINGS

Further advantages and features of the present invention will become apparent from the following detailed description of the invention when considered with reference to the accompanying drawings in which:

FIG. 1 is a graphic plot of typical FT-ICR MS data for two types of a crude oil sample solution prepared as described below;

FIG. 2 is a block diagram of a method in which an embodiment of the invention is implemented;

FIG. 3 is a schematic block diagram of modules of an embodiment of the invention; and

FIG. 4 is a block diagram of a computer system in which an embodiment of the invention is implemented.

### DETAILED DESCRIPTION OF INVENTION

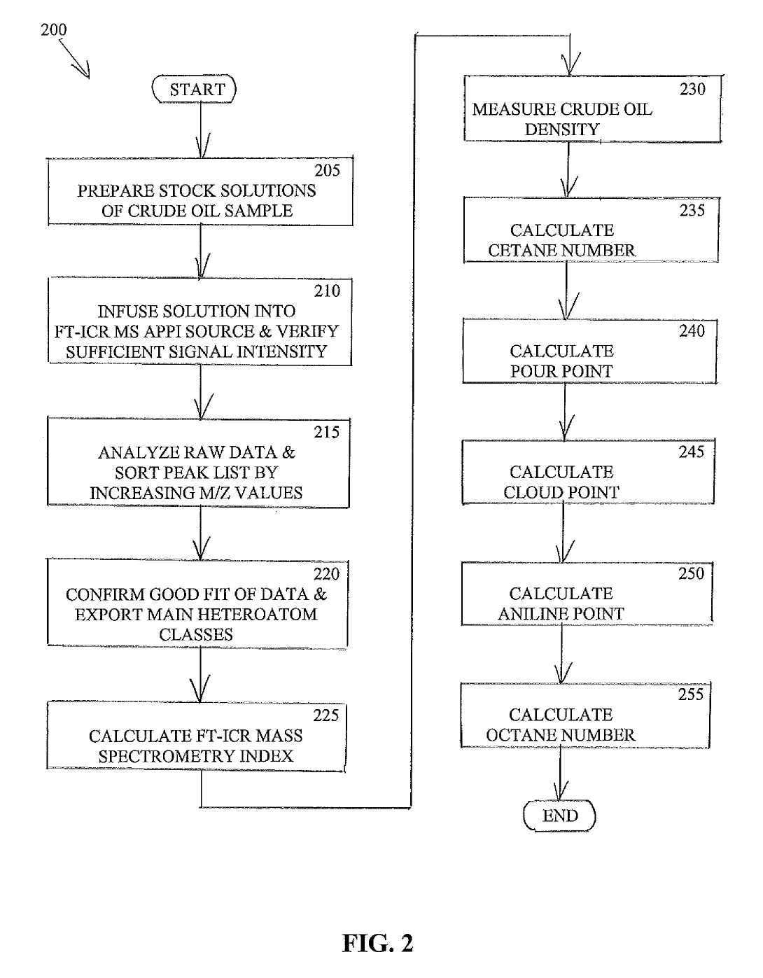

Crude oil samples were prepared and analyzed by atmospheric pressure photo ionization (APPI) Fourier transform ion cyclotron resonance mass spectrometry (FT-ICR MS) according to the method 200 described below, and illustrated in FIG. 2.

In step 205, Stock solution 1 is prepared by dissolving a 100 μL sample of the crude oil in 10 mL of toluene (or alternatively, in a 50/50% volume mixture of toluene with methanol, methylene chloride, dichloromethane or tetrahydrofuran). If complete solubility is not attained, based upon visual observation against a light source, methylene chloride is added to achieve a clear solution. The solution is shaken for a minimum of 20 seconds.

Solution 2 is prepared with a 1:100 dilution of solution 1 in methylene chloride. The miscibility of the solvent mix must be ensured.

Solution 3 is prepared with a 1:10 dilution of solution 2 in methylene chloride (i.e., 100 μL of solution 2 in 900 μL solvent).

The dilution ratio depends on the sample and has to be determined empirically on a case-by-case basis, starting from solution 3, then advancing to solution 2 and then to solution 1.

### Key Instrument Parameters

For each analysis of a sample, the operator tunes the spectrometer settings to optimize performance. Key parameters and default settings follow:

TD (Fid Size): 4M

Average Spectra: 100

Source Accumulation: 0.001 s

Ion Accumulation Time: 0.001 s

TOF (AQS): variable, depending on sample

APPI Temperature 250-400° C., depending on sample

Detection Mode: Broadband

Low Mass: 150 to 350 ink

High Mass: 3000m/z

### Mass Calibration and Performance Check

The performance of the FT-ICR MS instrument is checked by obtaining a mass calibration in EST positive mode. This ESI calibration can be used in the APPI mode by exchanging the EST ion source with the APPI source. The mass calibration remains valid for one day of normal operation as long as the key instrument parameters described above have not been changed. A change of any of the key instrument parameters requires a complete recalibration by switching to the ESI source, calibration, followed by switching back to the APPT source.

### Analysis

In step 210, the analysis begins with Solution 3, which is directly infused into the mass calibrated FT-ICR MS APPI source by a syringe pump. The operator records and averages 100 accumulated scans, which serve as a general basis for fine-tuning the instrument parameters.

If sufficient signal intensity (10⁸ to 10⁹ units) is not obtained with Solution 3, the analysis is repeated with Solution 2. If the analysis with Solution 2 still does not yield sufficient signal intensity, the analysis is repeated with Solution 1.

The operator checks the signal shape at the beginning, middle and end of the mass range. An excessive sample load can be diagnosed by a signal splitting. In case of signal splitting, all signals will appear as two closely aligned signals or, in severe cases, even as a group of signals. When the operator observes such signal splitting, he should dilute the sample until he obtains a good independent signal shape.

The following pass/fail criteria are applied to the tests. A mass calibration is acceptable when every mass calibrant in the mass range of the sample does not deviate more than ±0.2 ppm from the expected value, except calibrants that are discarded from the list due to either low intensity (below 3 times the baseline noise) or a calibrant signal that is overlapping a contamination signal.

### Data Processing Workflow

Data processing is an extensive exercise involving four different software packages as described below. Data processing can significantly impact the quality of the produced data and therefore must be performed by, or under the direction of an experienced scientist. The trade names of the respective programs are followed by their sources.

DataAcquisition from Bruker Daltonics of Bremen, Germany. The raw data is checked for sufficient signal shape and intensity as described above and, if necessary, re-measured until sufficient signal shape and intensity are obtained.

DataAnalysis from Bruker Daltonics of Bremen, Germany. The recorded raw data file is loaded into the DataAnalysis software. In step 215, the peak list is sorted according to increasing m/z values. The m/z values and intensities are then saved as a peak list “text file.”

Composer from SienaAnalytics of Modesto, Calif. The peak lists are loaded into the Composer software. The Composer software is started and a suitable parameter file is loaded. In step 220, the recalibration is checked by looking at the identified species. The individual series are inspected for consistency, i.e., for missing series and/or interrupted series, which may indicate non-ideal re-calibration. In exceptional cases, recalibration parameters have to be fine tuned until a good fit of the data is obtained. The main heteroatom classes, which are those constituting more than 1 percent of the assigned heteroatom classes, are exported into the Microsoft Excel spreadsheet “Automatic Processing Composer Data.xls.”

Excel Spreadsheet Automatic Processing Composer Data: This in-house developed spreadsheet processes the elemental compositions calculated by the Composer software and produces all graphs in a final reporting form. An Excel workbook with one summary tab and detail tabs for each identified heteroatom class is created.

Equation (1) shows the FT-ICR mass spectrometry index, FTMSI, which is calculated in step 225:

$$FTMSI = {\sum }_{C \# = min}^{max} ( Intensity ) / ( 1 E + 11 ) ; \qquad ( 1 )$$

where:

Intensity=the intensity for each carbon atom.

The indicative properties (i.e., the cetane number, pour point, cloud point and aniline point of the gas oil fraction boiling in the range 180-370° C. and octane number for gasoline fraction boiling in the range 36-180° C.) of the crude oil can be predicted from the density of whole crude oil (which is determined in step 230), and from the Fourier Transform Ion Cyclotron Resonance Mass Spectrometry index (FTMSI) of crude oil (which was determined in step 225). That is,

Indicative Property=f(density_{crude oil},FTMSI_{crude oil}) (2);

Equations (3) through (6) show, respectively, the cetane number, pour point, cloud point aniline point of gas oils boiling in the range 180-370° C., and equation (7) shows the octane number of gasoline boiling in the range 36-180° C. that can be predicted from the density and Fourier transform ion cyclotron resonance mass spectrometry index of crude oils. Thus, in step 235, the cetane number is calculated as:

Cetane Number(CET)=K_{CET}+X1_{CET}*DEN+X2_{CET}*FTMSI+X3_{CET}*FTMSI²+X4_{CET}*FTMSI³ (3);

In step 240, the pour point is calculated as:

Pour Point(PPT)=K_{PPT}+X1_{PPT}*DEN+X2_{PPT}*FTMSI+X3_{PPT}*FTMSI²+X4_{PPT}*FTMSI³ (4)

In step 245, the cloud point is calculated as:

Cloud Point(CPT)=K_{CPT}+X1_{CPT}*DEN+X2_{CPT}*FTMSI+X3_{CPT}*FTMSI²+X4_{CPT}*FTMSI³ (5)

In step 250, the aniline point is calculated as:

Aniline Point(AP)=K_{AP}+X1_{AP}*DEN+X2_{AP}*FTMSI+X3_{AP}*FTMSI²+X4_{AP}*FTMSI³ (6)

In step 255, the octane number is calculated as:

Octane Number(ON)=K_{ON}+X1_{ON}*DEN+X2_{ON}*FTMSI+X3_{ON}*FTMSI² (7)

where:

DEN=density of the crude oil sample;

FTMSI=Fourier transform ion cyclotron resonance mass spectrometry index (derived from FT-ICR MS data); and

K_{CET}, X1_{CET}-X4_{CET}, K_{PPT}, X1_{PPT}-X4_{PPT}, K_{CPT}, X1_{CPT}-X4_{CPT}, K_{AP}, X1_{AP}-X4_{AP}, K_{ON}, X1_{ON}-X3_{ON} are constants that were developed using linear regression analysis of hydrocarbon data from the APPI mode of FT-ICR MS, and which are given in Table 3.

TABLE 3

| | Cetane | Pour | Cloud | Aniline | Octane |
|---|---|---|---|---|---|
| Constants | Number | Point | Point | Point | Number |
| K | −322.2 | −266.1 | 4.5 | 166.7 | 128.8 |
| X1 | 419.0 | 299.4 | −3.4 | −119.8 | −91.1 |
| X2 | −22.9 | −180.7 | −127.2 | 51.0 | 8.8 |
| X3 | 198.8 | 558.1 | 330.6 | −123.9 | 3.2 |
| X4 | −175.3 | −387.4 | −215.0 | 70.2 | — |

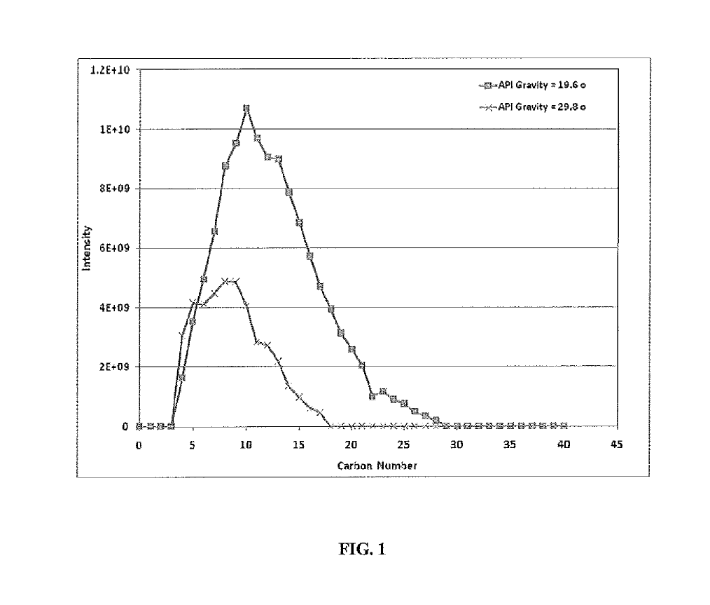

The following example is provided to demonstrate an application of equations (3) through (7). A sample of Arabian medium crude with a 15° C./4° C. density of 0.8828 Kg/I was analyzed by APPI FT-ICR MS, using the described method. The mass spectral data is presented in Table 4 and is shown in FIG. 1 as the sample with an API gravity of 28.8°.

The FT-ICR MS index, FTMSI, is calculated by summing the intensities of the detected peaks and then dividing by 1E+11, with the value in the example calculated as 0.40707.

TABLE 4

| Double Bond | |
|---|---|
| Equivalent | |
| (DBE) | Intensity |
| 0 | 0 |
| 1 | 0 |
| 2 | 0 |
| 3 | 0 |
| 4 | 3047754803 |
| 5 | 4148548475 |
| 6 | 4106580447 |
| 7 | 4475073884 |
| 8 | 4874039296 |
| 9 | 4852787148 |
| 10 | 4060232629 |
| 11 | 2831278701 |
| 12 | 2726027390 |
| 13 | 2196336212 |
| 14 | 1348225844 |
| 15 | 980497462 |
| 16 | 604773496 |
| 17 | 455374155 |
| 18 | 0 |
| 19 | 0 |

Applying equation (3) and the constants from Table 3,

Cetane Number(CET)=K_{CET}+X¹_{CET}*DEN+X2_{CET}*FTMSI+X3_{CET}*FTMSI²+X4_{CET}*FTMSI³=(−322.2)+(419.0)(0.8828)+(−22.9)(0.40707)+(198,8)(0.40707)²+(−175.3)(0.40707)³=59

Applying equation (4) and the constants from Table 3,

Pour Point(PPT)=K_{PPT}+X1_{PPT}*DEN+X2_{PPT}*FTMSI+X³_{PPT}*FTMSI²+X4_{PPT}*FTMSI³=(−266.1)+(299.4)(0.8828)+(−180.7)(0.40707)+(558.1)(0.40707)²+(−387.4)(0.40707)³−9

Applying equation (5) and the constants from Table 3,

Cloud Point(CPT)=K_{CPT}+X1_{CPT}*DEN+X2_{CPT}*FTMSI+X3_{CPT}*FTMSI²+X4_{CPT}*FTMSI³=(4.5)+(−3.4)(0.8828)+(−127.2)(0.40707)+(330.6)(0.40707)²+(−215.0)(0.40707)³=−10

Applying equation (6) and the constants from Table 3,

Aniline Point(AP)=K_{AP}+X1_{AP}*DEN+X2_{AP}*FTMSI+X3_{AP}*FTMSI²+X4_{AP}*FTMSI2=(166.7)+(−119.8)(0.8828)+(51.0)(0.40707)+(−123.9)(0.40707)²+(70.2)(0.40707)³=66

Applying equation (7) and the constants from Table 3,

Octane Number(ON)=K_{ON}+X1_{ON}*DEN+X2_{ON}*FTMSI+X3_{ON}*FTMSI²=(128.8)+(−91.1)(0.8828)+(8.8)(0.40707)+(3.2)(0.40707)²=52

The method is applicable for naturally occurring hydrocarbons derived from crude oils, bitumens, heavy oils, shale oils and from refinery process units including hydrotreating, hydroprocessing, fluid catalytic cracking, coking, and visbreaking or coal liquefaction.

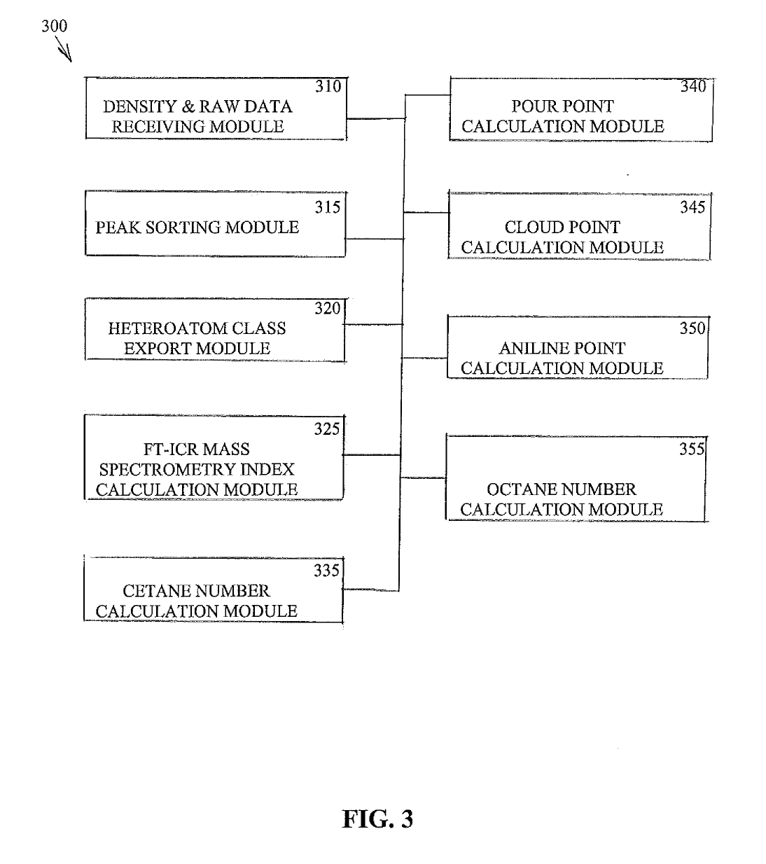

FIG. 3 illustrates a schematic block diagram of modules in accordance with an embodiment of the present invention, system 300. Density and raw data receiving module 310 receives Fourier transform ion cyclotron resonance mass spectrometry (FT-ICR MS) data derived from the corresponding crude oil and the density of a sample of crude oil. Peak sorting module 315 sorts the peaks by increasing m/z values. Heteroatom class export module 320 confirms a good fit of the FT-ICR MS data and uses the data to calculate the carbon numbers, double bond equivalents and intensities of the gas oil fraction. Module 330 calculates the FT-ICR mass spectrometry index (FTMSI). Cetane number calculation module 335 derives the cetane number for the gas oil fraction as a function of the FT-ICR MS peak intensity and density of the sample. Pour point calculation module 340 derives the pour point for the gas oil fraction as a function of the FT-ICR MS peak intensity and density of the sample. Cloud point calculation module 345 derives the cloud point for the gas oil fraction as a function of the FT-ICR MS peak intensity and density of the sample. Aniline point calculation module 350 derives the aniline point for the gas oil fraction as a function of the FT-ICR MS peak intensity and density of the sample. Octane number calculation module 355 derives the octane number for the gasoline fraction as a function of the FT-ICR MS peak intensity and density of the sample.

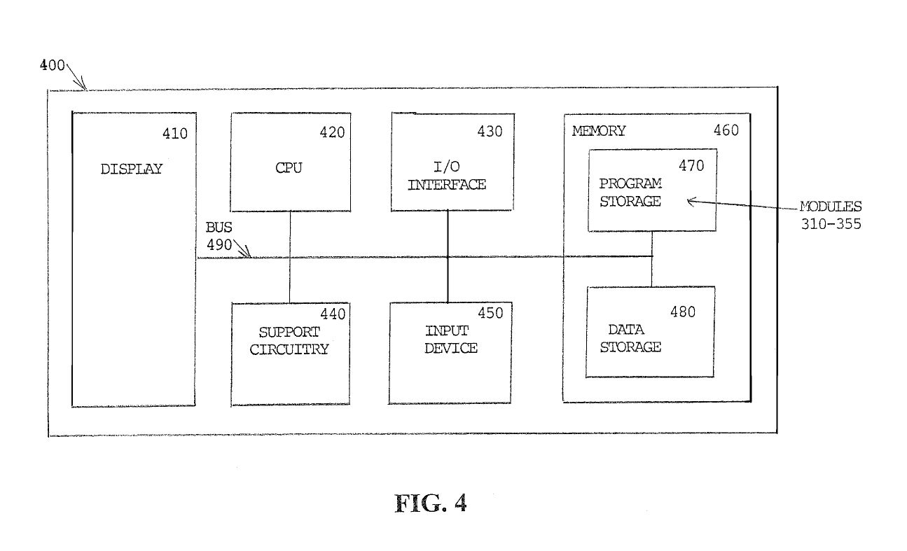

FIG. 4 shows an exemplary block diagram of a computer system 400 in which the partial discharge classification system of the present invention can be implemented. Computer system 400 includes a processor 420, such as a central processing unit, an input/output interface 430 and support circuitry 440. In certain embodiments, where the computer system 400 requires a direct human interface, a display 410 and an input device 450 such as a keyboard, mouse or pointer are also provided. The display 410, input device 450, processor 420, and support circuitry 440 are shown connected to a bus 490 which also connects to a memory 460. Memory 460 includes program storage memory 470 and data storage memory 480. Note that while computer system 400 is depicted with direct human interface components display 410 and input device 450, programming of modules and exportation of data can alternatively be accomplished over the input/output interface 430, for instance, where the computer system 400 is connected to a network and the programming and display operations occur on another associated computer, or via a detachable input device as is known with respect to interfacing programmable logic controllers.

Program storage memory 470 and data storage memory 480 can each comprise volatile (RAM) and non-volatile (ROM) memory units and can also comprise hard disk and backup storage capacity, and both program storage memory 470 and data storage memory 480 can be embodied in a single memory device or separated in plural memory devices. Program storage memory 470 stores software program modules and associated data, and in particular stores a density and raw data receiving module 310, peak sorting module 315, heteroatom class export module 320, FTMSI calculation module 325, cetane number calculation module 330, pour point calculation module 340, cloud point calculation module 345, aniline point calculation module 350, and octane number calculation module 355. Data storage memory 480 stores results and other data generated by the one or more modules of the present invention.

It is to be appreciated that the computer system 400 can be any computer such as a personal computer, minicomputer, workstation, mainframe, a dedicated controller such as a programmable logic controller, or a combination thereof. While the computer system 400 is shown, for illustration purposes, as a single computer unit, the system can comprise a group of computers which can be scaled depending on the processing load and database size.

Computer system 400 preferably supports an operating system, for example stored in program storage memory 470 and executed by the processor 420 from volatile memory. According to an embodiment of the invention, the operating system contains instructions for interfacing computer system 400 to the Internet and/or to private networks.

One of ordinary skill in the art will also comprehend that an embodiment of the partial discharge classification method of the present invention can be provided in the form of a computer program product.

The system and method of the present invention have been described above and with reference to the attached figure; however, modifications will be apparent to those of ordinary skill in the art and the scope of protection for the invention is to be defined by the claims that follow.

We claim:

1. A system for determining indicative properties of gasoline and gas oil fractions of crude oil, based upon Fourier transform ion cyclotron resonance mass spectrometry (FT-ICR MS) data derived from the corresponding crude oil and the density of the sample, the system comprising:
a non-volatile memory device that stores calculation modules and data;
a processor coupled to the memory;
a first calculation module that calculates the carbon numbers, double bond equivalents and intensities of the gas oil fraction from the FT-ICR MS data;
a second calculation module that derives the cetane number for the gas oil fraction as a function of the FT-ICR MS peak intensity and density of the sample;
a third calculation module that derives the pour point for the gas oil fraction as a function of the FT-ICR MS peak intensity and density of the sample;
a fourth calculation module that derives the cloud point for the gas oil fraction as a function of the FT-ICR MS peak intensity and density of the sample;
a fifth calculation module that derives the aniline point for the gas oil fraction as a function of the FT-ICR MS peak intensity and density of the sample; and
a sixth calculation module that derives the octane number for the gasoline fraction as a function of the FT-ICR MS peak intensity and density of the sample.

2. The system of claim 1, wherein the gas oil boils in the nominal range 180° C.-370° C.

3. The system of claim 1, wherein the gasoline boils in the nominal range 36° C.-180° C.

4. The system of claim 1, wherein the masses covered by FT-ICR MS are in the range 150-1400 m/z.

5. The system of claim 1, wherein the carbon numbers detected by FT-ICR MS are in the range 1-60.

6. The system of claim 1, wherein the double bond equivalence calculated by FT-ICR MS are in the range 1-40.

7. A method for operating a computer to determine indicative properties of gasoline and gas oil fractions of crude oil based upon a sample of the crude oil taken from an oil well, stabilizer, extractor, or distillation tower, the method comprising:
obtaining the density of the crude oil sample;
preparing the crude oil sample for Fourier transform ion cyclotron resonance mass spectrometry (FT-ICR MS) analysis;
obtaining spectra data for the crude oil sample by a FT-TCR MS analysis;
entering into the computer mass spectral data obtained by FT-ICR MS analysis of the crude oil sample;
calculating FT-ICR MS peak intensities of the gas oil fraction from the FT-ICR MS spectral data;
calculating the cetane number for the gas oil fraction as a function of the FT-ICR MS
calculating the pour point for the gas oil fraction as a function of the FT-ICR MS peak intensities and density of the sample;
calculating the cloud point for the gas oil fraction as a function of the FT-ICR MS peak intensity and density of the sample;
calculating the aniline point for the gas oil fraction as a function of the FT-ICR MS peak intensities and density of the sample; and
calculating the octane number for the gasoline fraction as a function of the FT-TCR MS peak intensity and density of the sample.

8. A computer program product to determine indicative properties of gasoline and gas oil fractions of crude oil based upon a sample of the crude oil taken from an oil well, stabilizer, extractor, or distillation tower, comprising a non-transitory computer readable medium having computer readable program code embodied therein that, when executed by a processor, causes the processor to:
accept the value of the density of the crude oil sample;
accept mass spectral data obtained by Fourier transform ion cyclotron resonance mass spectrometry (FT-ICR MS) analysis of the crude oil sample;
calculate FT-ICR MS peak intensities of the gas oil fraction from the FT-ICR MS spectral data;
calculate the cetane number for the gas oil fraction as a function of the FT-ICR MS peak intensities and density of the sample;
calculate the pour point for the gas oil fraction as a function of the FT-ICR MS peak intensities and density of the sample;
calculate the cloud point for the gas oil fraction as a function of the FT-ICR MS peak intensity and density of the sample;
calculate the aniline point for the gas oil fraction as a function of the FT-ICR MS peak intensities and density of the sample;
calculate the octane number for the gasoline fraction as a function of the FT-ICR MS peak intensity and density of the sample; and
display the calculated results and/or store the calculated results into memory.
